(19) United States
(12) Patent Application Publication
(10) Pub. No.: US 2008/0057552 A1
(43) Pub. Date: Mar. 6, 2008

(54) Processes for Producing Fats or Oils and Compositions Comprising the Fats or Oils

(76) Inventors: Inmok Lee, Decatur, IL (US); Lori Wicklund, Argenta, IL (US)

Correspondence Address: ARCHER DANIELS MIDLAND COMPANY, 4666 FARIES PARKWAY, DECATUR, IL, 62526, US

(21) Appl. No.: 11/847,895

(22) Filed: Aug. 30, 2007

Related U.S. Application Data
(60) Provisional application No. 60/841,669, filed on Aug. 31, 2006.

(51) Int. Cl. C12P 7/64; A23L 1/00; B01J 8/00

(52) U.S. Cl. 435/134; 422/129; 426/531

(57) ABSTRACT

Fats and oils subjected to modification by enzyme catalysts are pretreated with a granular clay, a combination of granular clay and protein, or a combination of granular clay and granular carbon resulting in improved productivity of the enzyme catalysts when used to modify the fats and oils.

### CROSS-REFERENCE TO RELATED APPLICATIONS

This application claims the benefit of U.S. Provisional Application No. 60/841,669, filed Aug. 31, 2006, the contents of the entirety of which is incorporated by this reference.

### TECHNICAL FIELD

The present invention relates generally to processes for producing fats and oils, as well as products produced from the processes.

### BACKGROUND

Lipases (triacylglycerol acylhydrolases, EC 3.1.1.3) are able to catalyze a variety of reactions. Such enzymes are commercially available from a broad range of manufacturers and organisms, and are useful in catalyzing reactions with commodity oils and fats. See, e.g., Xu, X., “Modification of oils and fats by lipase-catalyzed interesterification: Aspects of process engineering,” in Enzymes in Lipid Modification, 190-215 (Bornscheuer, U. T., ed., Wiley-VCH Verlag GmbH, Weinheim, Germany, 2000). Lipases are useful to hydrolyze glycerides such as triacylglycerols and phosphatides. They are also useful in the synthesis of esters from industrial fatty acids and alcohols. In addition, lipases are useful for alcoholysis (exchanging alcohols bound to esters) for products such as biodiesel and partial glycerides. Lipases can also be used to catalyze acyl-exchange reactions such as interesterification (also known as transesterification) of mixed ester substrates to create unique blends of triacylglycerols with desired functional characteristics.

Biocatalysts such as lipases are also attractive due to their use under mild operating conditions and their high degrees of selectivity. Biocatalysts also offer synthetic routes which avoid the need for environmentally harmful chemicals.

Lipases are further useful for the manufacture of specialty glycerides. For example, 1,3-specific lipases are useful in the manufacture of 1,3-diglycerides, as described, for example, in U.S. Pat. No. 6,004,611.

The transesterification reaction has also become an important solution to a recently identified threat to human health: trans fatty acids. These trans fatty acids were long desired for their functional characteristics in food use and have been produced on commodity scale by partial hydrogenation of vegetable oils. Thus, they have been readily available and relatively inexpensive for decades. Currently, suppliers of food products are seeking fats to replace partially hydrogenated vegetable oil, preferably at comparable prices or lower.

Transesterification of properly selected fats and oils can provide fats to replace partially hydrogenated vegetable oil. If such fats are produced by transesterification of fats and oils free from trans fatty acids, trans fatty acids will be substantially absent from the transesterified fat. Proper selection of fatty acid compositions of starting fats and oils will provide proper functionality in the transesterified replacement fats for partially hydrogenated oil advantageously synthesized by lipase-catalyzed interesterification.

The stability of biocatalysts such as lipases is most conveniently expressed in terms of half-life, which is the time after which the initial catalyst activity has decreased to half the original value. Diks, Rob M. M., “Lipase stability in oil,” Lipid Technology, 14(1): 10-14 (2002). Another way to express enzyme stability is the productivity of the enzyme, which is measured by the amount of the product per unit enzyme (g oil produced/g enzyme), during the first half-life. Typical lipase half-lives in interesterification reactions are seven days. See, e.g., Huang, Fang-Cheng and Ju, Yi-Hsu, “Interesterification of palm midfraction and stearic acid with Rhizopus arrhizus lipase immobilized on polypropylene,” Journal of the Chinese Institute of Chemical Engineers, 28(2): 73-78 (1997); Van der Padt, A. et al., “Synthesis of triacylglycerols. The crucial role of water activity control,” Progress in Biotechnology, 8 (Biocatalysis in Non-Conventional Media): 557-62 (1992). Half-lives vary greatly depending on the lipases themselves.

However, half-lives also vary depending on the quality of the substrates. When biocatalysts such as enzymes are used, components in the substrate mixture may diminish the effective lifetime of the catalyst. In continuous operations, the ratio of substrate processed to enzyme is very large, so minor components of oil can have a cumulative deleterious effect on enzyme activity. Several oxidation compounds in oil, such as hydroperoxides and secondary oxidation products (e.g., aldehydes or ketones), may cause significant lipase inactivation in oils. See, e.g., Pirozzi, Domenico, “Improvement of lipase stability in the presence of commercial triglycerides,” European Journal of Lipid Science and Technology 105(10): 608-613 (2003); Gray, J. I., “Measurement of Lipid Oxidation: A Review,” J. Amer. Oil Chem. Soc. 55: 539-546 (1978); U.S. Patent Application Publication No. 2005/0014237 A1, and publications cited therein. Oxidation products include oxidative species that initiate self-propagated radical reaction pathways, or other reactive oxygen species (such as peroxides, ozone, superoxide, etc.). These and other constituents which cause or arise from fat or oil degradation can result in enzyme degradation. The presence of water and other substances can also strongly influence the activity of lipases used in transesterification. See, e.g., Jung, H. J. and Bauer, W., “Determination of process parameters and modeling of lipase-catalyzed transesterification in a fixed bed reactor,” Chemical Engineering & Technology, 15(5): 341-8 (1992). Some metal ions (Mg 2+ and Fe 2+) have also been cited as inhibitors for some lipases. However, the processes and causative factors by which lipases become inactive are not completely understood.

It has been observed that using different batches of the same feedstock in a lipase-catalyzed reaction gave wide variations in lipase half-life. Diks, Rob M. M., “Lipase stability in oil,” Lipid Technology, 14(1): 10-14 (2002). No relationship was found between lipase half-life and the oil's PV or the para-anisidine value (PAV). In addition, no correlation between metal levels (Fe and Cu), polymerized glycerides, or phospholipids and lipase half-life could be established.

An investigation into the cause of loss of activity of immobilized lipase in the acidolysis of high oleic sunflower oil with stearic acid determined that oxidation products increased the rate of deactivation, but removal of oxidation products from the oils prevented activity loss. Nezu, T. et al., “The effect of lipids oxidation on the activity of interesterification of triglyceride by immobilized lipase,” in Dev. Food Eng., 6th Proc. Int. Congr. Eng. Food, 591-3 (Yano, T. et al., eds., Blackie, Glasgow, 1994). Immobilized lipases incubated with 2-unsaturated aldehydes (typically formed as secondary oxidation products in the oxidative breakdown of oils) lost their catalytic activity. Linoleic acid hydroperoxides at levels of PV>5 meq/kg causes loss of lipase activity, and the rate of enzyme inactivation increases as PV increased; the mechanism of enzyme inactivation was the generation of free radicals in the enzyme as the peroxides decomposed. Wang, Y. and Gordon, M. H., “Effect of lipid oxidation products on the transesterification activity of an immobilized lipase,” Journal of Agricultural and Food Chemistry, 39(9): 1693-5 (1991). When oxidized lipids were separated from a sample of palm oil and fractionated, it was demonstrated that fractions exhibiting high degrees of inactivation could be isolated, but the inhibitory compounds were not identified. Id.

Rapid lipase activity decrease during continuous lipase catalyzed reactions is common. See, e.g., Ferreira-Dias, S. et al. “Recovery of the activity of an immobilized lipase after its use in fat transesterification,” Progress in Biotechnology, 15 (Stability and Stabilization of Biocatalysis): 435-440 (1998); Diks, Rob M. M., “Lipase stability in oil,” Lipid Technology, 14(1):10-14 (2002).

Several methods have been tried to eliminate loss of activity or to recover activity from inactivated lipase.
a) Recovery of lipase activity lost in transesterification reactions was carried out by washing the lipase preparation with hexane and adjusting the water activity of the preparation to 0.22. Ferreira-Dias, S. et al. “Recovery of the activity of an immobilized lipase after its use in fat transesterification,” Progress in Biotechnology, 15 (Stability and Stabilization of Biocatalysis): 435-440 (1998). Although the mechanism was unknown, this type of activity recovery is consistent with activity loss caused by accumulation of inhibitory compounds such as lipid oxidation products. Id.
b) Reducing the water activity of a transesterification substrate (crude palm oil/degummed rapeseed oil) from 280 ppm to 60 ppm was accompanied by an increase of immobilized lipase half-life from 10 hours to 100 hours. Huang, Fang-Cheng and Ju, Yi-Hsu, “Interesterification of palm midfraction and stearic acid with Rhizopus arrhizus lipase immobilized on polypropylene,” Journal of the Chinese Institute of Chemical Engineers, 28(2):73-78 (1997).
c) Lipase half life has been increased by immobilizing certain compositions with lipase. For example, the half life of lipase immobilized on controlled pore silica increased fivefold when PEG-1500 was co-immobilized with the lipase. Soares, C. M. F. et al., “Selection of stabilizing additive for lipase immobilization on controlled pore silica by factorial design,” Applied Biochemistry and Biotechnology, 91-93(Symposium on Biotechnology for Fuels and Chemicals, 2000):703-718 (2001).
d) JP 11-103884 described the addition of small amounts (0.01-5 wt %) of phospholipids to an immobilized Alcaligenes lipase caused a ten-fold increase in lipase half life.
e) Others have prolonged lipase half-life via pre-treatment of the substrate oil. JP 08-140689 A2 describes the use of Duolite A-7 ion exchange resin to treat a blend of palm oil with ethyl stearate prior to interesterification using and immobilized Rhizopus lipase to increase the half life from 3 days to 8 days. Duolite A-7 is an anion exchange resin containing amino groups. JP 08-140689 A2 also describes pre-treatment of substrate oils with proteins or peptides containing a large number of basic amino acid residues such as histone, protamine, lysozyme or polylysine. JP 08-140689 A2 states that amino groups are believed to react with aldehydes or ketones (secondary oxidation products) to form a Schiff base; and that such secondary oxidation products are believed to be a factor in lipase inactivation.
f) JP 02-203789 A2 describes extending the half life of immobilized lipase by pre-treatment of the substrate with an alkaline substance. When an equal mixture of rapeseed oil and palm olein was interesterified on a column of lipase immobilized on Celite 535, the half life of the lipase was 18 hours. When the substrate was mixed with a solution of potassium hydroxide (5 mL/kg substrate) the half life of the enzyme activity was 96 h. An alternative approach is to treat celite with sodium hydroxide and mix this into the same substrate mixture. Using this approach, lipase half life was extended to 33 hours. JP 02 203790 A2.
g) It has been demonstrated that, Novozyme 435 is more affected by secondary oxidation products than by hydroperoxides (Pirozzi, Domenico, “Improvement of lipase stability in the presence of commercial triglycerides,” European Journal of Lipid Science and Technology 105(10):608-613 (2003)). With this lipase, it has been shown that lipase sulphydryl groups interact with two secondary oxidation product aldehydes, 4-hydroxynonenal (4-HNE) and malondialdehyde (MDA). By neutralizing 4-HNE and MDA in oil with albumin, enzyme stability was increased.
h) U.S. Patent Application No. 2003/0054509 describes the use of unmodified purification media (e.g., silica gel) to increase enzymatic half-life. U.S. Patent Application No. 2005/0014237 describes the use of deodorization processes to increase enzymatic half-life.
i) U.S. Pat. No. 5,288,619 teaches the passage of feed oil through a column containing absorbent clay or quinone-containing phenolic resin to absorb peroxides, oxygenated impurities, other similar species to prolong the half-life of enzymes used to treat the oil. The clay was further defined as “bleaching clay used to remove natural oil color compounds and enzyme poisons.”

Hence, there is a long-felt need in the art of enzymatic catalysis for solutions to this activity loss. See also Diks, Rob M. M., “Lipase stability in oil,” Lipid Technology, 14(1):10-14 (2002); Wang, Y. and Gordon, M. H., “Effect of lipid oxidation products on the transesterification activity of an immobilized lipase,” Journal of Agricultural and Food Chemistry, 39(9):1693-5 (1991). The time period over which lipase retains its enzymatic activity is an important cost consideration in lipase-catalyzed interesterification. The loss of effective enzyme activity is detrimental to industrial processing due to the cost of replacement enzyme and production time needed to change enzymes, switch columns, and stabilize a new column. Thus, the extension of enzyme half-life is extremely critical for the successful commercialization of enzymatic interesterification. This long-felt need is a primary barrier to the expansion of enzyme catalyzed reactions for production of commodity or “bulk” chemicals.

Although most of the mechanisms of lipase inactivation and its prevention are poorly understood at present, the present approach describes an effective solution to preventing lipase degradation and increasing its productivity and half-life.

### SUMMARY OF THE INVENTION

In one embodiment, a process for producing fats or oils comprises placing a glyceride in contact with a granular clay, thus forming a purified substrate, and placing the purified substrate in contact with a lipase, thus procuring the fat or the oil.

In another embodiment, a process for producing fats or oils comprise placing a glyceride in contact with a combination of a textured protein and a granular clay, thus producing a purified substrate, and contacting the purified substrate with a lipase, thus producing the fats or oils.

In an additional embodiment, a process for producing fats or oils comprises placing a glyceride in contact with a protein containing compound, a granular clay, or a combination thereof, thus generating a purified substrate, and contacting the purified substrate with lipase, thus producing the fats or the oils.

In a further embodiment, a process for producing fats or oils comprises placing glyceride in contact with a textured protein, a granular clay, or a combination thereof, thus generating a purified substrate, and contacting the purified substrate with lipase.

In yet another embodiment, a process comprises placing a lipid in contact with a food grade granular clay, thus producing a treated lipid.

In another embodiment, a process for producing fats or oils comprises placing a glyceride in contact with a granular clay and a granular carbon, thus forming a purified substrate, and placing the purified substrate in contact with a lipase, thus procuring the fat or the oil. are selected from the group consisting of edible substances, beverages, proteins, fats, oil, carbohydrates, supplements, nutrients, nutraceuticals, vitamins, medicines, food processing streams, food raw materials, animal feed, foodstuffs, mints, chewing gum, chewing tobacco, and combinations of any thereof.

In an embodiment of the invention, a combination of granular clay and granular carbon is used as a purification medium. Thus, an embodiment of the invention is directed to a method for producing fats or oils comprising contacting an initial substrate comprising one or more glycerides with one or more types of granular clay and one or more types of granular carbon to generate a purified substrate; and contacting the purified substrate with lipase to effect esterification, interesterification or transesterification creating the fats or oils.

In another embodiment of the invention, a method for producing fats or oils can also include monitoring enzymatic activity by measuring one or more physical properties of the fats or oils after having contacted the lipase; adjusting the duration of time for which the purified substrate contacts the lipase, or adjusting the temperature of the initial substrate, the purified substrate, the one or more types of purification media or the lipase in response to a change in the enzymatic activity to produce fats or oils having a substantially uniform increased proportion of esterification, interesterification, or transesterification relative to the initial substrate; and/or adjusting the amount and type of the one or more types of purification media in response to changes in the physical properties of the fats or oils to increase enzymatic productivity of the lipase. The one or more physical properties can include the Mettler dropping point temperature of the fats or oils and/or the solid fat content profile of the fats or oils.

In various methods, the initial substrate can also include any of free fatty acids, monohydroxyl alcohols, polyhydroxyl alcohols, esters and combinations thereof.

The one or more glycerides used in the inventive methods can include without limitation butterfat, cocoa butter, cocoa butter substitutes, illipe

In another embodiment, a system for treating a lipid comprises a container configured to place a lipid in contact with a substance capable of extending a half-life of an enzyme. The container comprises the substance capable of extending the half-life of the enzyme and the lipid.

A further embodiment is directed towards a product produced by a process comprising placing an ingestible substance in contact with a granular clay selected from the group consisting of granular clay, granular clay suitable for contact with human food products, food grade granular clay, food-compatible granular clay, granular clay approved for use in the production of human food products, a combination of granular clay and protein, a combination of granular clay and granular carbon, and any combinations thereof.

### BRIEF DESCRIPTION OF THE DRAWINGS

FIG. 1 shows the effects of passing a substrate mixture comprising refined, bleached, deodorized fully hydrogenated palm kernel oil through granular clay beds of varying volume before passing through a lipase column as in example 3 as used in one embodiment of the present invention. A control without granular clay pretreatment was also run.

### DETAILED DESCRIPTION OF THE INVENTION

Embodiments of the invention are directed to various methods for producing fats or oils. In one embodiment, an initial substrate comprising one or more glycerides is contacted with one or more types of purification media to generate a purified substrate, and the purified substrate is contacted with lipase to effect esterification, interesterification or transesterification creating the fats or oils. In one embodiment, the purification medium can be granular clay. In another embodiment, the purification media can be a combination of granular clay and textured vegetable protein. In another embodiment, the purification media can be a combination of granular clay and granular carbon.

In one embodiment, the granular clay may be a food grade granular clay that is suitable for use in human food products or approved for use in the production of human food products.

In an embodiment of the invention, granular clay is used as a purification medium. Thus, an embodiment of the invention is directed to a method for producing fats or oils comprising contacting an initial substrate comprising one or more glycerides with one or more types of granular clay to generate a purified substrate; and contacting the purified substrate with lipase to effect esterification, interesterification or transesterification creating the fats or oils.

In an embodiment of the invention, a combination of granular clay and vegetable protein is used as a purification medium. Thus, one embodiment is directed to a method for producing fats or oils comprising contacting an initial substrate comprising one or more glycerides with one or more types of granular clay and one or more types of vegetable protein to generate a purified substrate; contacting the purified substrate with lipase to effect esterification, interesterification or transesterification creating the fats or oils. In one embodiment of the invention, the vegetable protein can be a textured vegetable protein such as a textured soy protein. The enzymatic activity half-life of the lipase can be more than about 2.5 times greater than the enzymatic activity half-life resulting from contacting the lipase with the initial substrate.

In still yet another embodiment, the invention is directed towards use of a granular clay as a purification medium. Thus, one embodiment of the invention is directed to a method for producing fats or oils comprising contacting an initial substrate comprising one or more glycerides with one or more types of granular clay to generate a purified substrate; contacting the purified substrate with lipase to effect esterification, interesterification or transesterification creating the fats or oils.

In still yet another embodiment, the invention is directed towards use of a granular clay as a purification medium. Thus, one embodiment of the invention is directed to a method for treating foodstuffs, food, or ingestible substances comprising contacting an initial substrate comprising one or more ingestible substance with one or more types of granular clay to generate a purified ingestible substance. In various embodiments, ingestible substances fat, kokum butter, milk fat, mowrah fat, phulwara butter, sal fat, shea fat, borneo tallow, lard, lanolin, beef tallow, mutton tallow, tallow, animal fat, canola oil, castor oil, coconut oil, coriander oil, corn oil, cottonseed oil, hazelnut oil, hempseed oil, jatropha oil, linseed oil, mango kernel oil, meadowfoam oil, mustard oil, neat's foot oil, olive oil, palm oil, palm kernel oil, peanut oil, rapeseed oil, rice bran oil, safflower oil, sasanqua oil, shea butter, soybean oil, sunflower seed oil, tall oil, tsubaki oil, vegetable oils, marine oils which can be converted into plastic fats, marine oils which can be converted into solid fats, menhaden oil, candlefish oil, cod-liver oil, orange roughy oil, pile herd oil, sardine oil, whale oils, herring oils, 1,3-dipalmitoyl-2-monooleine (POP), 1(3)-palmitoyl-3(1)-stearoyl-2-monooleine (POSt), 1,3-distearoyl-2-monooleine (StOSt), triglycerides, diglycerides, 1,3-diglycerides, monoglycerides, behenic acid triglyceride, triolein, tripalmitin, tristearin, palm olein, palm stearin, palm kernel olein, palm kernel stearin, triglycerides of medium chain fatty acids, or combinations thereof; processed partially hydrogenated oils of any of the foregoing; processed fully hydrogenated oils of any of the foregoing; fractionated oils of any of the foregoing partially hydrogenated soybean oil, partially hydrogenated corn oil, partially hydrogenated cottonseed oil, fully hydrogenated soybean oil, fully hydrogenated corn oil, partially hydrogenated palm oil, partially hydrogenated palm kernel oil, fully hydrogenated palm oil, fully hydrogenated palm kernel oil, fractionated palm oil, fractionated palm kernel oil, fractionated partially hydrogenated palm oil, fractionated partially hydrogenated palm kernel oil; and any combinations thereof.

In an additional embodiment, the initial substrate can also include esters. The esters can be any of wax esters, alkyl esters, methyl esters, ethyl esters, isopropyl esters, octadecyl esters, aryl esters, propylene glycol esters, ethylene glycol esters, 1,2-propanediol esters, 1,3-propanediol esters, and combinations thereof. The esters can be formed from the esterification or transesterification of monohydroxyl alcohols or polyhydroxyl alcohols. The monohydroxyl alcohols or the polyhydroxyl alcohols can be primary, secondary or tertiary alcohols of annular, straight or branched chain compounds. The monohydroxyl alcohols can be any of methyl alcohol, isopropyl alcohol, allyl alcohol, ethanol, propanol, n-butanol, iso-butanol, sec-butanol, tert-butanol, n-pentanol, iso-pentanol, n-hexanol or octadecyl alcohol. The polyhydroxyl alcohols can be any of glycerol, propylene glycol, ethylene glycol, 1,2-propanediol, 1,3-propanediol, trimethylol propane, pentaerythritol, and sugars.

The initial substrate can also have primary, secondary or tertiary monohydroxyl alcohols of annular, straight or branched chain compounds. The monohydroxyl alcohols can be any of methyl alcohol, isopropyl alcohol, allyl alcohol, ethanol, propanol, n-butanol, iso-butanol, sec-butanol, tert-butanol, n-pentanol, iso-pentanol, n-hexanol or octadecyl alcohol.

The initial substrate used in the inventive methods can also have primary, secondary or tertiary polyhydroxyl alcohols of annular, straight or branched chain compounds. The polyhydroxyl alcohols can be any of glycerol, propylene glycol, ethylene glycol, 1,2-propanediol or 1,3-propanediol.

The initial substrate can also have one or more fatty acids which are saturated, unsaturated or polyunsaturated. The one or more fatty acids can have carbon chains from about 4 to about 22 carbons long. The fatty acids can be any of palmitic acid, stearic acid, oleic acid, linoleic acid, linolenic acid, arachidonic acid, erucic acid, caproic acid, caprylic acid, capric acid, lauric acid, myristic acid, eicosapentaenoic acid (EPA), docosahexaenoic acid (DHA), 5-eicosenoic acid, behenic acid, butyric acid, alpha-linolenic acid, gamma-linolenic acid, conjugated linoleic acid or any combination thereof.

In another embodiment, the one or more types of purification media and the lipase are packed in one or more columns. The columns can be jacketed columns in which the temperature of the initial substrate, the purified substrate, the one or more types of purification media or the lipase is regulated.

In other embodiments, the purified substrate can be prepared by mixing the initial substrate with the one or more types of purification media in a tank for a batch slurry purification reaction or mixing the initial substrate in a series of tanks for a series of batch slurry purification reactions. The purified substrate can be separated from the one or more types of purification media via filtration, centrifugation or concentration prior to reacting the purified substrate with the lipase. The purified substrate can be mixed with the lipase in a tank for a batch slurry reaction, or flowing the purified substrate through a column containing the lipase.

In yet other embodiments, a bed of the one or more types of purification media is placed upon a bed of the lipase within a column. The column can be a jacketed column in which the temperature of the initial substrate, the purified substrate, the one or more types of purification media or the lipase is regulated.

The lipase can be obtained from a cultured eukaryotic or prokaryotic cell line. The lipase can be a 1,3-selective lipase or a non-selective lipase. The fats or oils produced can be 1,3-diglycerides.

The one or more glycerides used in the methods of the invention can be partially hydrogenated soybean oil, partially hydrogenated corn oil, partially hydrogenated cottonseed oil, fully hydrogenated soybean oil, fully hydrogenated corn oil, and/or fully hydrogenated cottonseed oil.

In other embodiments, the one or more glycerides used in the methods of the invention can be partially hydrogenated palm oil, partially hydrogenated palm kernel oil, fully hydrogenated palm oil, fully hydrogenated palm kernel oil, fractionated palm oil, fractionated palm kernel oil, fractionated partially hydrogenated palm oil, and/or fractionated partially hydrogenated palm kernel oil.

In other embodiments, the product of the lipase-catalyzed reaction can be used in embodiments including, but not limited to: food and supplements; beverages; cosmetics and personal care products; compositions for animal feed; frying oils; confectionary coatings and fillings; margarine oil or spread oil; release agents for pans, belts, molds etc.; baking fats for inclusion in bake products including, but not limited to, frozen dough, filling fats, dry mixes for baking, cookies, cream cakes, foam cakes, yeast-raised products, including without limitation breads, buns, rolls, fried bread; frozen foods; emulsions, including, but not limited to, sauces, creams, mayonnaise, toppings, yogurts; microwave popcorn oil; spray oil for baking, frying and cooking use; cheese; or combinations of any thereof. The oil of the present invention can also be blended with other oils or fats to provide a fat blend having desired characteristics or be blended with other foodstuffs to provide a food composition having desired characteristics.

The present invention relates to increasing the productivity or enzymatic half-life of enzymes that catalyze esterification, interesterification or transesterification. In particular, the present invention relates to the removal of constituents which cause lipase degradation from an initial substrate. Such constituents may cause or arise from fat or oil degradation, from substrate handling or processing, or from other causes. Such constituents can be removed by treating the initial substrate with a purification medium prior to contacting the lipase. The purification medium can be one or more granular clays, a combination of granular clay and granular carbon, or a combination of granular clay and vegetable protein such as textured vegetable protein.

Denaturation of the side chains of enzymes, especially at the active sites, is believed to be a cause of the loss of enzyme activity. The denaturation can be caused by reactions between the amino acid side chains on the enzyme and substrate impurity constituents which cause enzyme degradation. However, different enzymes have different amino acid side chains involved in enzyme denaturation. Hence, the present invention includes screening granular clays, proteins, and granular carbons for their ability to react with substrate impurity constituents and hence serve as an initial substrate purification media to increase enzymatic half-life. Such screening can also be done with initial substrate which contains the substrate impurity constituents.

In another embodiment, the present invention includes using granular clay, textured protein, or granular carbon for initial substrate purification where it is known that one or more particular granular clay constituent, protein constituent, or granular carbon constituent are prone to reacting with or binding to substrate impurities where the reactions between the substrate impurities and the enzyme result in inactivating the enzyme. Thus, granular clay, textured proteins, or granular carbon can have a protective effect for enzymes by functioning as a “trap” to react with and/or remove inactivating compounds in the substrates, preventing the enzymes from being denatured by the compounds. Trapping of the inactivating compounds may also provide a means to concentrate the inactivating compounds for recovery and use, such as use as selective enzyme inactivators.

Clay has utility in treating vegetable oils as “bleaching clay”. These clays are derived from clay mineral deposits which are dried, milled, sieved, and possibly activated with acid. One common type of clay found on most continents is Bentonite. Most Bentonite includes forms of aluminum silicate known as Montmorillonite, which is characterized by a three-layer structure. A central sheet of alumina in an octahedral structure is sandwiched between two layers of silica in a tetrahedral structure. Electrochemical binding attracts the sheets together. Magnesium ions are often incorporated into the alumina layer, and mobile cations such as Ca⁺², Na⁺ and K⁺ are often found between the layers. Clays containing predominantly Ca⁺² are classed as Bentonites, which are amenable to activation by treatment with acid. Several companies supply bleaching clays, including, but not limited to, Sud-Chemie Inc (Louisville, Ky.), LaPorte Absorbents (Cheshire, England), and Engelhard Corp. (Beachwood, Ohio). Bleaching clays are very fine powders, typically having 90% of particles below 80 microns in diameter, and substantially all particles less than 200 micron particles. A typical particle size distribution for bleaching clay is given in Table AA.

TABLE AA
Typical size distribution for bleaching clay (from “Bleaching
and Purifying Fats and Oils. Theory and Practice”, H. B. W.
Patterson, AOCS Press, Champaign, 1992, p. 170 & 223).

| Size (microns) | Percentage (by weight) |
|---|---|
| Greater than 80 | 10 |
| 40-80 | 25 |
| 20-40 | 30 |
| Less than 20 | 35 |

For ease of reference, the unaided eye can distinguish individual particles down to 40 microns; thus, the bleaching clay universally used in bleaching fats and oils is very fine.

Granular clay has conventionally been used in non-food applications. For example, KH series granular clay from Zhejiang Anji Zhongxin Activated Clay Company, Ltd. (Gaoyu, Anji, Zhejiang, China) is used for refining aviation kerosene and solvents, such as by removing alkenes. Clayton Fullers Earth Granular Clay from Mahajeet Clayton Industries (Mumbai, India) is suitable for bleaching paraffin oil, transformer oil, and other highly viscous fluids, and treating light kerosene. Granular absorbent clay is routinely used in cleaning up spills and drips in car bays, machine shops, warehouses, factory floors, packaging plants, septic tanks, abattoirs, garages, farms, and zoos, such as MultiZorb from Abzorboil (Cleobury Mortimer, Kidderminster, UK). In addition, granular clay can be used in cat litter. These non-food granular clays are formed into granules which are conventionally dried in a kerosene-fired or oil-fired furnace. Substances which are potentially toxic by ingestion are generated in the combustion gases from these fuels, and may condense on the granular clay. This renders most commercial granular clay unsuitable for food contact uses.

An embodiment of granular clay useful in the embodiments of the present invention includes special-run UltraClear 30/60 Adsorbent from OilDri, Corp. (Vernon Hills, Ill.). In conventional use (that is, not the special run material) this product is dried during production with an oil-fired furnace and used for clarification of jet fuel. Another embodiment of granular clay useful in embodiments of the present invention include special-run Agsorb 30/60 Adsorbent from OilDri, Corp. (Vernon Hills, Ill.). In conventional use (that is, not the special run material), this product is dried during production with an oil-fired furnace and is used for field distribution of agricultural chemicals. Special run material is identical except that it is dried using natural gas to produce a food grade granular clay having a moisture content below 2% and suitable for food use in that the drying process used to produce the food grade granular clay does not result in a substantial condensation of toxic gases on the food grade granular clay. The particle size distribution of the food grade granular clay is given in Table BB.

TABLE BB
Particle size distribution of special run food grade granular clay

| Size (mesh) | Size (microns) | Percentage (by weight) |
|---|---|---|
| Less than 20 | Greater than 625 | 1.6 |
| 20-60 | ˜240-625 | 97.0 |
| 60-100 | ˜150-˜240 | 1.1 |
| Greater than 100 | Less than ˜150 | 0.3 |

The present invention also relates to using textured protein as a substrate purification medium. The protein can be textured vegetable protein (for example, textured soy protein) and/or other proteins, such as whey protein. In particular, the present invention is directed to using such a protein to purify the initial substrate prior to contacting the substrate with lipase. In one embodiment, textured vegetable protein is used. Textured vegetable protein has a rigid texture and an expanded, open structure which provides greater surface area to interact with oil, thus conferring substantial advantages over conventional protein in its use for oil treatment.

Amino-groups in conventional peptides or proteins (such as those described in JP 08-140689 A2) are bound and not readily available to react with secondary oxidation products. In a non-aqueous medium such as vegetable oil, ionic forces holding proteins together tend to be at least an order of magnitude greater than other forces in the media (e.g., van der Waals interactions or hydrogen bonding). Conventional proteins in a non-aqueous matrix tend to clump together and present the smallest possible total surface area to the non-aqueous medium. Thus, conventional proteins minimize the amino groups available for interaction with the oil components believed to cause enzyme inactivation. Hence, amino acids of conventional proteins are relatively impenetrable (and unavailable) to oils and other non-aqueous media, and do not as readily react with the oil components believed to cause enzyme inactivation.

The proteins used in the present invention possess advantages over conventional proteins. In one embodiment, TVP® brand textured vegetable protein available from Archer-Daniels-Midland Company of Decatur, Ill. is used. The moisture content of this product is typically about 6%. Advantages conferred by the texturizing process include, but are not limited to, particle rigidity and increased surface area relative to the untextured protein. Other treatments such as typical soybean expanders and collet forming devices may also be used to confer desired properties on protein.

Good contact between the initial substrate and a granular clay or protein substrate purification medium can be facilitated by using a protein which is relatively dry. Thus, in one embodiment, the moisture content of the protein (for example a vegetable protein or a textured vegetable protein) is less than about 5%. For example, the moisture content of the protein can be from about 0% to about 5%, or any amount between about 0% and about 5% (e.g. about 0%, about 1%, about 2%, about 3%, about 4%, or about 5%), or any range between about 0% and about 5% (e.g. about 2% to about 4%).

The moisture range of the protein (for example a vegetable protein or a textured vegetable protein) can be controlled during manufacture to give the desired moisture content. The moisture content of the protein can also be adjusted after manufacture such as, for example, by oven drying or contact with a solvent that removes some of the moisture from the textured vegetable protein. Moisture can be removed by other known methods including without limitation by washing with anhydrous solvents. For example, the moisture content of textured vegetable protein containing 6% moisture can be reduced by washing with anhydrous ethanol. Ethanol-washed textured vegetable protein can be rinsed with a solvent that has good miscibility with triacylglycerols, such as acetone, ethyl acetate, or hexane.

The typical composition of the soybean is about 18% oil, about 38% protein, about 15% insoluble carbohydrate (dietary fiber), about 15% soluble carbohydrate (sucrose, stachyose, raffinose, others) and about 14% moisture, ash and other. See, e.g., Egbert, W. R., “Isolated soy protein: Technology, properties, and applications,” in Soybeans as Functional Foods and Ingredients, 134-163 (KeShun L., ed., AOCS Press, Champaign, Ill. 2004). Textured soy protein may be made by first cracking soybeans to remove the hull and rolling the beans into full-fat flakes. The rolling process disrupts the oil cell, facilitating solvent extraction of the oil. The solvent is removed and the flakes are dried, creating defatted soy flakes. The defatted flakes can be ground to produce soy flour, sized to produce soy grits or texturized to produce textured soy protein such as Archer-Daniels-Midland Company's TVP® brand textured vegetable protein. The defatted flakes can be further processed to produce soy protein concentrates and isolated soy protein. This is accomplished by the removal of the carbohydrate components of the soybean followed by drying.

Soy proteins are generally classified into three groups: soy flours, soy protein concentrates and isolated soy proteins with minimum protein contents of about 50%, about 65% and about 90% (dry basis), respectively. Soy flours are sold as either fine powders or grits with a particle size ranging from ˜0.2 to 5 mm. These products can be manufactured using minimal heat to maintain the inherent enzyme activity of the soybean, or lightly to highly toasted to reduce or eliminate the active enzymes. Soy flours and grits have been traditionally used as an ingredient in the bakery industry.

Soy protein concentrates are traditionally manufactured using aqueous-alcohol to remove the soluble sugars from the defatted soy flakes (soy flour). This process results in a protein with low solubility and a product that can absorb water, but lacks the ability to gel or emulsify fat.

Traditional alcohol washed concentrates are used for protein fortification of foods as well as in the manufacture of textured soy protein concentrates. Functional soy protein concentrates bind water, emulsify fat and form a gel upon heating. Functional soy protein concentrates can be produced from alcohol-washed concentrate using heat and homogenization followed by spray-drying; or produced using a water-wash process at an acidic pH to remove the soluble sugars followed by neutralization, thermal processing, homogenization and spray-drying. Functional soy protein concentrates are widely used in the meat industry to bind water and emulsify fat. These proteins are also effective in stabilizing high fat soups and sauces.

Textured or structured soy proteins can be made from soy flour, soy protein concentrate or isolated soy protein. TVP® brand textured vegetable protein is manufactured through thermoplastic extrusion of soy flour under moist heat and high pressure. The skilled artisan is familiar with the varieties of textured vegetable protein. Textured soy protein concentrate is produced from soy protein concentrate powders using similar manufacturing technology to Archer-Daniels-Midland Company's TVP® brand textured vegetable protein. Unique textured protein products can be produced using combinations of soy protein or other powdered protein ingredients such as wheat gluten in combination with various carbohydrate sources (e.g. starches). The skilled artisan is familiar with the textured products manufactured by thermoplastic extrusion technology. Such products are distributed in dry form throughout the world. These products are hydrated in water or flavored solutions prior to usage in processed meat products, vegetarian analogs or used alone in other finished food products to simulate meat. Spun fiber technology can be used to produce a fibrous textured protein from isolated soy protein with a structure closely resembling meat fibers.

Isolated soy proteins can be manufactured from defatted soy flakes by separation of the soy protein from both the soluble and insoluble carbohydrate of the soybean.

One soy protein suitable for use in the present invention includes, without limitation, Archer-Daniels-Midland Company's TVP® brand textured vegetable protein (Decatur, Ill.). Such soy protein is a product of commerce containing nominally about 53% protein, about 3% fat, about 18% total dietary fiber, about 30% carbohydrates and about 9% maximum moisture. This material is available in a variety of textures, sizes and colors and is used in the food industry as a substitute for ground meat in beef patties, sausage, vegetarian foods, meatloaf mix and other similar food applications. One product that may be used is Archer-Daniels-Midland Co. product code 165 840, which is supplied as pale yellow granules of about 1/16 inch diameter.

Soy protein manufactured according to other processes is also useful in the present invention. For example, the soy protein can also be the textured vegetable proteins described in U.S. Pat. Nos. 4,103,034 and 4,153,738, which are hereby incorporated by reference.

Granular carbons can be prepared from natural carbon sources, such as coal. An embodiment of a granular carbon is Cal®12×40 Granular bituminous coal-based carbon from Calgon Carbon Corporation.

The present invention also relates to using an unmodified purification medium to reduce the constituents which cause or arise from fat or oil degradation within the fat or oil substrate. Accordingly, one method of making an esterified, transesterified or interesterified product can further comprise contacting the initial substrate (i.e., fats or oils alone, or mixed with additional components such as esters, free fatty acids or alcohols) with one or more types of unmodified purification media, thus producing a purification media-processed substrate. The purification media can contact the substrate in one or more columns or in one or more batch slurry type reactions. The purification medium can come into contact with the substrate before the substrate comes into contact with the enzyme. Any of the purification media and methods of use described in U.S. Patent Application Publication No. 2003/0054509 A1 can be used in combination with the present invention, and are hereby incorporated herein by reference.

Deodorization can be used in combination with the purification techniques described by the present invention. Examples of deodorization processes include, but are not limited to, the deodorization techniques described by O. L. Brekke, Deodorization, in Handbook of Soy Oil Processing and Utilization, Erickson, D. R. et al. eds., pp. 155-191 published by the American Soybean Association and the American Oil Chemists' Society; or by Bailey's Industrial Oil and Fat Products, 5th ed., Vol. 2 (pp. 537-540) and Vol. 4 (pp. 339-390), Hui, Y. H. ed., published by John Wiley and Sons, Inc. Deodorization at ambient temperature can also be used as it will remove air (which can cause oxidation of oil) from oil. Other deodorization processes that may be used include, without limitation, those described in U.S. Pat. Nos. 6,172,248 and 6,511,690; and in U.S. Patent Application Publication No. 2005/0014237 A1. All of these deodorization techniques are hereby incorporated by reference. In one embodiment, the pretreatment methods of the present approach obviate the need for deodorization of substrate before contacting with the lipase.

The present invention also contemplates preventing oxidation of the substrate oil by keeping the oil under an inert gas. Inert gases that may be used include, but are not limited to, nitrogen, carbon dioxide, helium, or any combination thereof which may be used during or after purification. The esterified, transesterified or interesterified products of the present invention can also be deodorized after the treatment with enzyme.

For purposes herein, the term “initial substrate” includes, but is not limited to, refined or unrefined, bleached or unbleached and/or deodorized or non-deodorized fats or oils. The fats or oils can comprise a single fat or oil or combinations of various fats or oils. According to the present invention, a substrate can be recycled (i.e., deodorized, contacted with purification media, esterified, transesterified or interesterified more than once). Hence, the skilled artisan would recognize that “initial substrate” includes, without limitation: substrates that have never been deodorized; substrates that have been deodorized one or more times; substrates that have never contacted purification media; substrates that have contacted purification media one or more times; substrates that have never been esterified, transesterified or interesterified; and/or substrates that have been esterified, transesterified or interesterified one or more times. The esterification, transesterification or interesterification process may be catalyzed enzymatically, such as with a lipase, or chemically, such as with alkali or alkoxide catalysts.

The terms “purification media-processed substrate” or “purified substrate” include a substrate which has contacted one or more purification media at least once. Prior to its contact with enzyme, an initial substrate or a purification media-processed substrate can be mixed with additional components including esters, free fatty acids or alcohols. These esters, free fatty acids or alcohols which are added to the initial substrate or purification media-processed substrate can optionally contact purification media prior to contacting enzyme.

The terms “product” and “esterified, transesterified or interesterified product” are used interchangeably and include esterified, transesterified or interesterified fats, oils, triglycerides, diglycerides, monoglycerides, mono- or polyhydroxyl alcohols, or esters of mono- or polyhydroxyl alcohols produced via an enzymatic transesterification or esterification process. The term “product” as used herein, has come into contact at least once with an enzyme capable of causing esterification, transesterification or interesterification. A product can be a fluid or solid at room temperature, and is increased in its proportional content of esterified, transesterified or interesterified fats, oils, triglycerides, diglycerides, monoglycerides, mono- or polyhydroxyl alcohols, or esters of mono- or polyhydroxyl alcohols as a result of its having contacted the transesterification or esterification enzyme. Esterified, transesterified or interesterified product is to be distinguished from the contents of initial substrate or purification-media processed substrate, in that product has undergone additional enzymatic transesterification or esterification reaction. The present invention includes use of any combination of the deodorization, purification and transesterification or esterification processes for the production of esterified, transesterified or interesterified fats, oils, triglycerides, diglycerides, monoglycerides, mono- or polyhydroxyl alcohols, or esters of mono- or polyhydroxyl alcohols.

The term “enzyme” as used herein includes but is not limited to lipases, as discussed herein, or any other enzyme capable of causing modifying fats or oils, such as by esterification, transesterification or interesterification of substrate. Other enzymes capable of modifying fats and oils include but are not limited to oxidoreductases, peroxidases, and esterases.

Fats and oils include triglycerides made up of a glycerol backbone in which the hydroxyl groups are esterified with carboxylic acids. Whereas solid fats tend to be formed by triglycerides having saturated fatty acids, triglycerides with unsaturated fatty acids tend to be liquid (oils) at room temperature. Monoglycerides and diglycerides, having respectively one fatty acid ester and two alcoholic groups or two fatty acid esters and one alcoholic group, are also found in fats and oils to a lesser extent than triglycerides. which is part of the initial substrate, or from a free fatty acid or ester that has been added to the initial substrate or purification media-processed substrate.

The term “esterification” includes the process in which R, R′ or R″ on a glyceride is converted from an alcoholic group (OH) to a fatty acid group given by —OC(═O)R′″. The fatty acid group which replaces the alcoholic group can come from the same or different glyceride, or from a free fatty acid or ester that has been added to the initial substrate or the purification media-processed substrate. The present invention also includes esterification of alcohols which have been added to the initial substrate or the purification media-processed substrate. For example, an alcohol so added may be esterified by an added free fatty acid or by a fatty acid group present on a glyceride which was a component of the initial substrate. A non-limiting example of esterification includes reaction of a free fatty acid with an alcohol.

Esterification also includes processes pertaining to the manufacture of biodiesel, such as discussed in U.S. Pat. Nos. 5,578,090; 5,713,965; and 6,398,707, which are hereby incorporated by reference. The term “biodiesel” includes, but is not limited to, lower alkyl esters of fatty acid groups found on animal or vegetable glycerides. Lower alkyl esters include without limitation methyl ester, ethyl ester, n-propyl ester, and isopropyl ester. In the production of biodiesel, the initial substrate comprises fats or oils. One or more lower alcohols (e.g., methanol, ethanol, n-propanol and isopropanol) are added to this substrate and the mixture comes into contact with enzyme. The enzyme causes the alcohols to be esterified with the fatty acid groups which is part of the fat or oil glycerides. For example, R, R′ or R″ on a glyceride is a fatty acid group given by —OC(═O)R′″. Upon esterification of methanol, the biodiesel product is CH3OC(═O)R′″. Biodiesel products also include esterification of lower alcohols with free fatty acids or other esters which are added to the initial substrate or purification media-processed substrate.

The term “transesterification” includes the process in which R, R′ or R″ on a glyceride is a first fatty acid group given by —OC(═O)R′″, and the first fatty acid group is replaced by a second, different fatty acid group. The second fatty acid group which replaces the first fatty acid group can come from the same or different fat or oil present in the initial substrate. The second fatty acid can also come from a free fatty acid or ester added to the initial substrate or the purification media-processed substrate. The present invention also includes transesterification or interesterification of esterified alcohols or other esters which have been added to the initial substrate or the purification media-processed substrate. For example, an alcohol so added may be transesterified or interesterified by an added free fatty acid, by a fatty acid group on an added ester, or by a fatty acid group present on a glyceride which was a component of the initial substrate. A non-limiting example of transesterification includes reaction of a fat or oil with an alcohol (e.g., methanol) or with an ester.

The term “interesterification” includes, for example, the processes acidolysis, alcoholysis, glycerolysis, and transesterification. Examples of these processes are described herein, and in Rousseau, D. and Marangoni, A. G., “Chemical Interesterification of Food Lipids: Theory and Practice,” in Food Lipids Chemistry, Nutrition, and Biotechnology, Second Edition, Revised and Expanded, Akoh, C. C. and Min, D. B. eds., Marcel Dekker, Inc., New York, N.Y., Chapter 10, which is hereby incorporated by reference. Acidolysis includes the reaction of a fatty acid with an ester, such as a triacylglycerol; alcoholoysis includes the reaction of an alcohol with an ester, such as a triacylglycerol; and glycerolysis includes alcoholysis reactions in which the alcohol is glycerol. A non-limiting example of interesterification or transesterification includes reactions of different triglycerides resulting in rearrangement of the fatty acid groups in the resulting glycerides and triglycerides.

An esterified, transesterified or interesterified product has respectively undergone the esterification, transesterification or interesterification process. The present invention relates to enzymes capable of effecting the esterification, transesterification or interesterification process for fats, oils, triglycerides, diglycerides, monoglycerides, free fatty acids, mono- or polyhydroxyl alcohols, or esters of mono- or polyhydroxyl alcohols.

As used herein, the “half-life” of an enzyme is the time in which the enzymatic activity of an enzyme sample is decreased by half. If, for “acid groups” attached to the glycerides or to other esters used as substrates in the present invention. That is, a substrate of the present invention can comprise fats, oils or other esters having fatty acid groups formed from the free fatty acids or fatty acids discussed herein.

The one or more unrefined and/or unbleached fats or oils can comprise butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow or other animal fat, canola oil, castor oil, coconut oil, coriander oil, corn oil, cottonseed oil, hazelnut oil, hempseed oil, Jatropha oil, linseed oil, mango kernel oil, meadowfoam oil, mustard oil, neat's foot oil, olive oil, palm oil, palm kernel oil, palm olein, palm stearin, palm kernel olein, palm kernel stearin, peanut oil, rapeseed oil, rice bran oil, safflower oil, sasanqua oil, soybean oil, sunflower seed oil, tall oil, tsubaki oil, vegetable oils, marine oils which can be converted into plastic or solid fats such as menhaden oil, candlefish oil, cod-liver oil, orange roughy oil, pile herd oil, sardine oil, whale and herring oils, 1,3-dipalmitoyl-2-monooleine (POP), 1(3)-palmitoyl-3(1)-stearoyl-2-monooleine (POSt), 1,3-distearoyl-2-monooleine (StOSt), triglycerides, diglycerides, monoglycerides, behenic acid triglyceride, triolein, tripalmitin, tristearin, triglycerides of medium chain fatty acids, or combinations thereof.

Processed fats and oils such as hydrogenated or fractionated fats and oils can also be used. Examples of fractionated fats include, without limitation, palm olein, palm stearin, palm kernel olein, palm kernel stearin, and combinations of any thereof. Fully or partially hydrogenated, saturated, unsaturated or polyunsaturated forms of the above listed fats, oils, triglycerides or diglycerides are also useful for the present invention. The described fats, oils, triglycerides or diglycerides are usable singly, or at least two of them can be used in admixture.

“Esterification” or “transesterification” are the processes by which a fatty acid group is added, repositioned or replaced on one or more components of the substrate. The acid group can be derived from a fat or oil example, an enzyme sample decreases its relative activity from 100 units to 50 units in 10 minutes, then the half life of the enzyme sample is 10 minutes. If the half-life of this sample is constant, then the relative activity will be reduced from 100 to 25 in 20 minutes (two half lives), the relative activity will be reduced from 100 to 12.5 in 30 minutes (three half lives), the relative activity will be reduced from 100 to 6.25 in 40 minutes (four half lives), etc. As used herein, the expression “half-life of an enzyme” means includes the half-life of an enzymatic sample.

A “prolonged” half-life refers to an increased “half-life”. Prolonging the half-life of an enzyme results in increasing the half life of an enzyme by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%,135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 320%, 340%, 360%, 380%, 400%, 420%, 440%, 460%, 480%, 500% or more as compared to the half-life of an enzyme used in an esterified, transesterified or interesterified fat or oil producing process which does not employ a purification medium.

Non-limiting examples of “constituents which cause or arise from fat or oil degradation” include without limitation oxidative or oxidating species, reactive oxygen species, fat or oil oxidation products, peroxides, ozone (O3), O2, superoxide, free fatty acids, volatile organic compounds, free radicals, trace metals, and natural prooxidants such as chlorophyll. Such constituents also include other characterized or uncharacterized compounds recognized by the skilled artisan to cause or arise from fat or oil degradation. Such constituents can arise from oxidation pathways, or from other pathways recognized by the skilled artisan to result in fat or oil degradation. “Reducing” the constituents which cause or arise from fat or oil degradation in a substrate sample refers to lowering the concentration, percentage or types of such constituents in the sample.

A method of making an esterified, transesterified or interesterified product of the present invention can further comprise mixing the initial substrate and/or the purification media-processed substrate with the enzyme in one or more tanks for a batch slurry reaction, or flowing the initial substrate and/or the purification media-processed substrate through a column containing the enzyme. A bed of the one or more types of purification media can be placed upon a bed of the enzyme within a column upstream from the enzyme.

In one embodiment, the initial substrate, the purification media-processed substrate, the esterified, transesterified or interesterified product and the enzyme can be in an inert gas environment. The inert gas can be selected from the group consisting of N2, CO2, He, Ar, Ne, and combinations thereof. The methods of the present invention may further comprise preventing oxidative degradation of the initial substrate, the purification media-processed substrate, the esterified, transesterified or interesterified product or the enzyme. The method of making an esterified, transesterified or interesterified product can further comprise preventing oxidative degradation to the initial substrate, the purification media-processed substrate, the esterified, transesterified or interesterified product or the enzyme.

The skilled artisan would recognize that in respect to the method of making an esterified, transesterified or interesterified product, any combination of the above described particulars pertaining to deodorization options (e.g., flow rate, residence or holding time, temperature, pressure, choice of inert gas), initial substrate, components (e.g., free fatty acids, non-glyceride esters, alcohols) optionally added to the initial substrate or the purification media-processed substrate, enzyme, monitoring or adjusting methods, fats or oils produced, use of columns or batch slurry reactions, and purification medium are useful in the present invention.

Transesterification, esterification or interesterification according to the present invention may be effected by a lipase. The lipase can be specific or unspecific with respect to its substrate. The initial substrate can include one or more types of fat or oil and have its physical properties modified in an esterification, transesterification or interesterification process. Nonselective enzymes cause rearrangement by transesterification at all three positions on a glyceride, and may result in randomization at thermodynamic equilibrium; but 1,3-specific lipases cause rearrangements at the sn-1 and sn-3 positions on a glyceride. For example, when a blend of olive oil and fully hydrogenated palm kernel oil is treated with a non-selective enzyme, the components of the product have different physical properties from either of the initial substrates. Both 1,3-specific lipases and nonselective lipases are capable of this rearrangement process.

The lipase may be a 1,3-selective lipase, which catalyzes esterification or transesterification of the terminal esters in the sn-1 and sn-3 positions of a glyceride. The lipase can also be a non-selective, nonspecific lipase. The process can produce esterified, transesterified or interesterified fats with no or reduced trans fatty acids for margarine, shortening, and other confectionery fats such as cocoa butter substitute. The esterified, transesterified or interesterified product can also be a 1,3-diglyceride, such as those disclosed in U.S. Pat. No. 6,004,611.

The enzyme used in one embodiment can be a lipase obtained from a cultured eukaryotic or prokaryotic cell line or animal tissue. Such lipases typically fall into one of three categories (Macrae, A. R., J.A.O.C.S. 60:243 A-246A (1983)). The first category includes nonspecific lipases capable of releasing or binding any fatty acid group from or to any glyceride position. Such lipases have been obtained from Candida cylindracae, Corynebacterium acnes and Staphylococcus aureus (Macrae, 1983; U.S. Pat. No. 5,128,251). The second category of lipases adds or removes specific fatty acid groups to or from specific glycerides. Thus, these lipases are useful in producing or modifying specific glycerides. Such lipases have been obtained from Geotrichum candiium and Rhizopus, Aspergillus, and Rhizomucor genera (Macrae, 1983; U.S. Pat. No. 5,128,251). The last category of lipases catalyze the removal or addition of fatty acid groups from the glyceride carbons on the end in the 1- and 3-positions. Such lipases have been obtained from Thermomyces lanuginosa, Rhizomucor miehei, Aspergillus niger, Mucor javanicus, Rhizopus delemar, and

Glycerides useful in the present approach include, without limitation, molecules of the chemical formula CH2RCHR′CH2R″ wherein R, R′ and R″ are alcohols (OH) or fatty acid groups given by —OC(═O)R′″, wherein R′″ is a saturated, unsaturated or polyunsaturated, straight or branched carbon chain with or without substituents. R, R′, R″ and the fatty acid groups on a given glyceride can be the same or different. The acid groups R, R′ and R″ can be obtained from any of the free fatty acids described herein. Glycerides for the present invention include triglycerides in which R, R′ and R″ are all fatty acid groups, diglycerides in which two of R, R′ and R″ are fatty acid groups and one alcohol functionality is present; monoglycerides in which one of R, R′ and R″ is a fatty acid group and two alcohol functionalities are present; and glycerol in which each of R, R′ and R″ is an alcohol group. Glycerides useful as starting materials in the present invention include, but are not limited to, natural fats and oils, processed fats and oils, refined fats and oils, refined and bleached fats and oils, refined, bleached and deodorized fats and oils, expelled fats and oils, synthetic fats and oils, and combinations of any thereof. The process can also be carried out on in the presence of a substrate in contact with a solvent. An example is soybean oil miscella, which is the product of solvent extraction of soybean oil and often comprises crude soybean oil in hexane. Examples of refined fats and oils are described herein and in Stauffer, C., Fats and Oils, Eagan Press, St. Paul, Minn. (1996). Examples of processed fats and oils are refined, refined and bleached, hydrogenated and fractionated fats and oils.

The terms “fatty acid groups” or “acid groups” both refer to chemical groups given by —OC(═O)R′″. Such “fatty acid groups” or “acid groups” are connected to the remainder of the glyceride via a covalent bond to the oxygen atom that is singly bound to the carbonyl carbon. In contrast, the terms “fatty acid” or “free fatty acid” both refer to HOC(═O)R′″ and are not covalently bound to a glyceride. In “fatty acid groups,” “acid groups,” “free fatty acids,” and “fatty acids,” R′″ is a saturated, unsaturated or polyunsaturated, straight or branched carbon chain with or without substituents, as described herein. The skilled artisan will recognize that R′″ of the “free fatty acids” or “fatty acids” (i.e., HOC(═O)R′″) described herein are useful as R′″ in the “fatty acid groups” or Rhizopus arrhizus (Macrae, 1983). Enzymes from animal sources, such as pig (Sus scrofa) pancreas lipase, can also be used.

There are many microorganisms from which lipases useful in the present invention may be obtained. U.S. Pat. No. 5,219,733 lists examples of such microorganisms including those of the genus Achromobacter such as A. iofurgus and A. lipolyticum; the genus Chromobacterium such as C. viscosum var. paralipolyticum; the genus Corynebacterium such as C. acnes; the genus Staphylococcus such as S. aureus; the genus Aspergillus such as A. niger and A. oryzae; the genus Candida such as C. cylindracea, C. antarctica b, C. rosa and C. rugosa; the genus Humicola such as H. lanuginosa and H. rosa; the genus Penicillium such as P. caseicolum, P. crustosum, P. cyclopium and P. roqueforti; the genus Torulopsis such as T. ernobii; the genus Mucor such as M. miehei, M. japonicus and M. javanicus; the genus Bacillus such as B. subtilis; the genus Thermomyces such as T. ibadanensis and T. lanuginosa (see Zhang, H. et al. JAOCS 78: 57-64 (2001)); the genus Rhizopus such as R. delemar, R. japonicus, R. arrhizus and R. neveus; the genus Pseudomonas such as P. aeruginosa, P. fragi, P. cepacia, P. mephitica var. lipolytica and P. fluorescens; the genus Alcaligenes; the genus Rhizomucor such as R. miehei; and the genus Geotrichum such as G. candidum. Several lipases obtained from these organisms are commercially available as purified enzymes. The skilled artisan would recognize other enzymes capable of affecting esterification, transesterification or interesterification including other lipases useful for the present invention.

Lipases obtained from the organisms described herein may be immobilized for the present invention on suitable carriers by a method known to persons of ordinary skill in the art. U.S. Pat. Nos. 4,798,793; 5,166,064; 5,219,733; 5,292,649; and 5,773,266 describe examples of immobilized lipase and methods of preparation. Examples of methods of preparation include the entrapping method, inorganic carrier covalent bond method, organic carrier covalent bond method, and the adsorption method. The lipase used in the exemplary embodiments herein were obtained from Novozymes (Denmark), but can be substituted with purified and/or immobilized lipases prepared by others manufacturers. The present invention also includes using crude enzyme preparations or cells of microorganisms capable of over expressing lipase, a culture of such cells, a substrate enzyme solution obtained by treating the culture, or a composition containing the enzyme. The present invention also includes the use of more than one enzyme preparation, such as more than one lipase preparation.

The esterification, transesterification or interesterification can be conducted in a column or in batch slurry type reactions as described in the exemplary embodiments herein. In the batch slurry reactions, the enzyme and substrates are mixed sufficiently to ensure a good contact between them, taking care not to mix under high shear, which could cause loss of enzyme activity. The transesterification or esterification reaction is carried out in a fixed bed reactor with immobilized lipases.

The fatty acid groups described herein can be added to the initial substrate or the purification media-processed substrate to esterify alcoholic groups present on glycerides of the initial substrate, or alcoholic groups of other compounds (e.g., alcohols or esters) added to the purification media-processed substrate. Glycerides having any of the fatty acid groups as described herein can also be used in the initial substrate; and other esters having any of the fatty acid groups described herein can be added to the initial substrate or purification media-processed substrate. Such fatty acids include saturated straight-chain or branched fatty acid groups, unsaturated straight-chain or branched fatty acid groups, hydroxy fatty acid groups, and polycarboxylic acid groups, or contain non-carbon substituents including oxygen, sulfur or nitrogen. The fatty acid groups can be naturally occurring, processed or refined from natural products or synthetically produced. Although there is no upper or lower limit for the length of the longest carbon chain in useful fatty acids, i their length may be about 6 to about 34 carbons long. Specific fatty acid groups useful for the present invention can be formed from the fatty acids described in U.S. Pat. Nos. 4,883,684; 5,124,166; 5,149,642; 5,219,733; and 5,399,728.

Examples of useful saturated straight-chain fatty acid groups having an even number of carbon atoms can be formed from the fatty acids described in U.S. Pat. No. 5,219,733 including, but not limited to, acetic acid, butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid and heptacosanoic acid.

Examples of useful saturated branched fatty acid groups can be formed from fatty acids described in U.S. Pat. No. 5,219,733 including, without limitation, isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, a-ethyl-hexanoic acid, a-hexyldecanoic acid, a-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.)

Examples of useful saturated odd-carbon branched fatty acid groups can be formed from fatty acids described in U.S. Pat. No. 5,219,733 including, but not limited to, anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid and 26-methyloctacosanoic acid.

Examples of useful unsaturated fatty acid groups can be formed from fatty acids described in U.S. Pat. No. 5,219,733 including, without limitation, 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-1-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, alpha-eleostearic acid, beta-eleostearic acid, punicic acid, 6,9,12,15-octadecatetraenoic acid, parinaric acid, 5,8,11,14-eicosatetraenoic acid, 5,8,11,14,17-eicosapentaenoic acid (EPA), 7,10,13,16,19-docosapentaenoic acid, 4,7,10,13,16,19-docosahexaenoic acid (DHA) and the like.

Examples of useful hydroxy fatty acid groups can be formed from fatty acids described in U.S. Pat. No. 5,219,733 including, but not limited to, α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like.

Examples of useful polycarboxylic acid fatty acid groups can be formed from fatty acids described in U.S. Pat. No. 5,219,733 including, without limitation, oxalic acid, citric acid, malonic acid, succinic acid, glutaric acid, adipic acid, pimelic acid, suberic acid, azelaic acid, sebacic acid, D,L-malic acid and the like.

In one embodiment, the fatty acid groups have carbon chains from about 4 to about 34 carbons long. In another embodiment, the fatty acid groups have carbon chains from about 4 to about 26 carbons long. In yet an additional embodiment, the fatty acid groups have carbon chains from about 4 to about 22 carbons long. The fatty acid groups are formed from the following group of free fatty acids: palmitic acid, stearic acid, oleic acid, linoleic acid, linolenic acid, arachidonic acid, erucic acid, caproic acid, caprylic acid, capric acid, eicosapentanoic acid (EPA), docosahexaenoic acid (DHA), lauric acid, myristic acid, 5-eicosenoic acid, butyric acid, alpha-linolenic acid, gamma-linolenic acid, and conjugated linoleic acid. Fatty acid groups formed from fatty acids derived from various plant and animal fats and oils (such as fish oil fatty acids) and processed or refined fatty acids from plant and animal fats and oils (such as fractionated fish oil fatty acids in which EPA and DHA are concentrated) can also be added. Fatty acid groups can also be formed from medium chain fatty acids (as described by Merolli, A. et al., INFORM, 8:597-603 (1997)). The fatty acid groups may be formed from free fatty acids having carbon chains from about 4 to about 36, about 4 to about 24, or about 4 to about 22 carbons long.

Alcohols or esters of alcohols can also be added to the initial substrate or the purification media-processed substrate. These alcohols and esters can be esterified, transesterified or interesterified by acid groups present on glycerides of the initial substrate. These alcohols or esters thereof may also be esterified, transesterified or interesterified by free fatty acids or esters added to the purification media-processed substrate. “Esters” include any of the alcohols described herein esterified by any of the fatty acids described herein.

Examples of useful esters other than glycerides include wax esters, alkyl esters such as methyl, ethyl, isopropyl, hexadecyl or octadecyl esters, aryl esters, propylene glycol esters, ethylene glycol esters, 1,2-propanediol esters and 1,3-propanediol esters. Esters can be formed from the esterification, transesterification or interesterification of monohydroxyl alcohols or polyhydroxyl alcohols by the free fatty acids, fats or oils as described herein.

The initial substrate or purification media-processed substrate can be mixed with monohydroxyl alcohols or polyhydroxyl alcohols prior to contact with the purification medium or the enzyme. The esterified, transesterified or interesterified product can be formed from the esterification, transesterification or interesterification of the monohydroxyl alcohols or polyhydroxyl alcohols. The monohydroxyl alcohols or the polyhydroxyl alcohols can be primary, secondary or tertiary alcohols of annular, straight or branched chain compounds. The monohydroxyl alcohols can be selected from the group consisting of methyl alcohol, isopropyl alcohol, allyl alcohol, ethanol, propanol, n-butanol, iso-butanol, sec-butanol, tert-butanol, n-pentanol, iso-pentanol, n-hexanol, hexadecyl alcohol, octadecyl alcohol and combinations of any thereof. The polyhydroxyl alcohols can be selected from the group consisting of glycerol, propylene glycol, ethylene glycol, 1,2-propanediol, 1,3-propanediol and combinations of any thereof.

Examples of alcohols useful in the present invention include monohydroxyl alcohols or polyhydroxyl alcohols. The monohydroxyl alcohols can be primary, secondary or tertiary alcohols of annular, straight or branched chain compounds with one or more carbons such as methyl alcohol, isopropyl alcohol, allyl alcohol, ethanol, propanol, n-butanol, iso-butanol, sec-butanol, tert-butanol, n-pentanol, iso-pentanol, n-hexanol, hexadecyl alcohol or octadecyl alcohol. The hydroxyl group can be attached to an aromatic ring, such as phenol. Examples of polyhydroxyl alcohols includes glycerol, propylene glycol, ethylene glycol, 1,2-propanediol and 1,3-propanediol.

U.S. Pat. No. 5,219,733 indicates other alcohols useful for the present invention. These alcohols include, but are not limited to, 14-methylhexadecanol-1, 16-methyloctadecanol-1, 18-methylnonadecanol, 18-methyleicosanol, 20-methylheneicosanol, 20-methyldocosanol, 22-methyltricosanol, 22-methyltetracosanol, 24-methylpentacosanol-1 and 24-methylhexacosanol.

The one or more types of purification media and the enzyme can be packed together or separately in one or more columns through which the initial substrate, the purification-media processed substrate or the esterified, transesterified or interesterified product flows. The columns can be jacketed columns in which the temperature of one or more of the initial substrate, the purification media-processed substrate, the one or more types of purification media, the enzyme or the esterified, transesterified or interesterified product can be regulated. The purification media-processed substrate can be prepared by mixing the initial substrate with the one or more types of purification media in a tank for a batch slurry purification reaction or mixing the initial substrate in a series of tanks for a series of batch slurry purification reactions. The purification media-processed substrate can be separated from the one or more types of purification media via filtration, centrifugation or concentration prior to reacting the purification media-processed substrate with the enzyme. The purification medium may be kept separate from the enzyme. By keeping the purification medium separate from the enzyme, the impurity constituents of the initial substrate which degrade lipase do not come into contact with the lipase.

In one method of the present invention, one or more types of purification media and the lipase may be packed into one or more columns. The purification medium may be kept separate from (i.e., not intermixed with) the active lipase. If multiple types of purification media are used, they can be mixed together and packed into a single column or kept separate in different columns. In an alternative embodiment, one or more types of purification media are placed upon a bed of packed lipase within a column. Alternatively, the active lipase can be kept separate from the purification media by packing it in its own column. More than one type of purification media can be used for purposes of removing different kinds of impurities in the initial substrate. The columns and other fluid conduits can be jacketed so as to regulate the temperature of the initial substrate, the purification media-processed substrate, the purification media or the enzyme. The purification media can be regenerated for repeated use.

Also in one method of the present invention, the purification media-processed substrate is prepared by mixing the initial substrate with one or more types of purification media in a tank for a batch slurry type purification reaction or mixing the initial substrate in a series of tanks for a series of batch slurry type purification reactions. In these batch slurry type purification reactions, the different types of purification media can be kept separate or can be combined. After reacting with one type of purification medium (or specific mixture of purification media), the initial substrate is separated from the purification medium (or media) via filtration, centrifugation or concentration. The initial substrate may be further purified with other purification media or serves as purification media-processed substrate and is reacted with lipase. The purification media-processed substrate prepared by this batch slurry type purification reaction method can be reacted with lipase in a tank for batch slurry type transesterification or esterification. Alternatively, the purification media-processed substrate can be caused to flow through a lipase column. The reacting tanks, columns and other fluid conduits can be jacketed so as to regulate the temperature of the initial substrate, the purification media-processed substrate, the purification media or the enzyme. Other manners of temperature regulation, such as heating/cooling coils or temperature controlled rooms, are contemplated and well known in the art. The purification media can be regenerated for repeated use.

Lipase enzymatic activity is also affected by factors such as temperature, light and moisture content. Temperature is controlled as described herein. Light can be kept out with various light blocking or filtering means known in the art. Moisture content, which includes ambient atmospheric moisture, is controlled by operating the process as a closed system. Where the process includes deodorization using steam as a stripping agent, the deodorization process can be kept isolated from the enzyme. Because deodorization is performed at high temperature and under vacuum, moisture content in the deodorized oil is very low. Where the deodorization process uses an inert gas as the stripping agent, the deodorization process is optionally kept isolated from the enzyme. Alternatively, a bed of nitrogen gas (or other inert gas) can be placed on top of the bed or column containing either purification medium or enzyme. These techniques have the added benefit of keeping atmospheric oxidative species (including oxygen) away from the substrate, product or enzyme.

Immobilized lipase can be mixed with initial substrate or purification media-processed substrate to form a slurry which is packed into a suitable column. Alternatively, substrate or purified substrate can flow through a pre-packed enzyme column. The temperature of the substrate is regulated so that it can continuously flow though the column for contact with the transesterification or esterification enzyme. If solid or very viscous fats, oils, triglycerides or diglycerides are used, the substrate is heated to a fluid or less viscous state. The substrate can be caused to flow through the column(s) under the force of gravity, by using a peristaltic or piston pump, under the influence of a suction or vacuum pump, or using a centrifugal pump. The transesterified fats and oils produced are collected and the desired glycerides are separated from the mixture of reaction products by methods well known in the art. This continuous method involves a reduced likelihood of permitting exposure of the substrates to air during reaction and therefore has the advantage that the substrates will not be exposed to moisture or oxidative species. Alternatively, reaction tanks for batch slurry type production as described herein can also be used. These reaction tanks may be sealed from air so as to prevent exposure to oxygen, moisture, or other ambient oxidizing species.

The method of the present invention may also comprise monitoring enzymatic activity by measuring one or more physical properties of the esterified, transesterified or interesterified product; and optionally adjusting the duration of time for which the purified substrate contacts the lipase, or adjusting the temperature of the initial substrate, the purified substrate, the one or more types of the purification medium or the lipase in response to a change in enzymatic activity, to produce fats or oils having a substantially uniform increased proportion of esterification, interesterification, or transesterification relative to the initial substrate as measured by physical properties. The duration of time for which the purified substrate contacts the lipase can be adjusted by adjusting the flow rate of purified substrate provided to contact with the lipase. Also, the amount and type of the one or more types of purification media can be adjusted in response to changes in the physical properties of the fats or oils to increase or improve enzymatic productivity of the lipase.

By the phrase “substantially uniform increased proportion of esterification, interesterification, or transesterification relative to the initial substrate,” it is meant that the amount or degree of esterification, interesterification, or transesterification of the oil or fat produced from a particular initial substrate by the methods of the invention varies by no more than about 10% in one embodiment, no more than about 5% in another embodiment as measured by a change in one of the physical property measurements as described herein.

In the present invention, changes in enzymatic activity are monitored by following changes in the physical properties of the product. As the enzymatic activity decreases, the rate of substrate conversion decreases so that less of the substrate is converted into product via esterification, transesterification or interesterification at a given flow rate than the initial amount of conversion. Consequently, as the enzymatic activity decays, the physical properties of the product increasingly resemble the physical properties of the components of the substrate. The skilled artisan recognizes that by following changes in physical properties, the parameters of the esterified, transesterified or interesterified production process can be adjusted, thus increasing the proportion of esterified, transesterified or interesterified product relative to the substrate, so that fats and oils with a desired degree of esterification, interesterification, or transesterification can be produced while improving the enzymatic productivity of the lipase.

The one or more physical properties of the fats or oils product that can be measured during the methods of the invention include without limitation the dropping point temperature of the product, the solid fat content profile of the product, changes in optical spectra, and combinations of any thereof.

The Mettler dropping point (MDP) is one example of a physical property which can be measured to follow changes in enzymatic activity. The MDP is determined using Mettler Toledo, Inc. (Columbus, Ohio) thermal analysis instruments according to the American Oil Chemists Society Official Method #Cc 18-80. The MDP is the temperature at which a mixture of fats or oils becomes fluid.

The product's solid fat content (SFC) profile (as a function of temperature) is another useful physical property for tracking changes in enzymatic activity. SFC can be measured according to American Oil Chemists Society Official Method #Cd 16b-93.

Following changes in optical spectra is another way to monitor changes in enzymatic activity. The substrate and product each have a characteristic optical spectrum. As the lipase activity decays, the amount of product that gives rise to spectroscopic signals attributable to esterified, transesterified or interesterified product (and not attributable to substrate) diminishes.

All of these properties are measured using techniques well known in the art, and are useful in following changes in enzymatic activity and for determining the uniformity of esterification, interesterification, or transesterification of the produced oils or fats.

For example, as the lipase enzymatic activity decays, less substrate is converted into product resulting in an increased substrate:product ratio. This increased ratio is manifested in a change of physical properties of the outflowing product tending towards the physical properties of the non-esterified or non-transesterified substrate. To minimize this change, the flow rate of the substrate is reduced so that it is exposed for a longer period of time to the packed lipase. The flow rate reduction increases the product:substrate ratio and, consequently, the physical properties of the outflowing fats or oils reflect that of the desired esterified, transesterified or interesterified product. Other process parameters that can be altered include the flow rate, temperature or pressure of the initial substrate or the purification media-processed substrate.

Where purification media-processed substrate is reacted with lipase in a tank for batch slurry type production, changes in the product's physical properties can also be monitored as described above. In a batch slurry type process, an optimized duration of time is determined for contacting the initial substrate with the purification medium (or media). An optimized time is also determined for contacting the purification media-processed substrate with enzyme.

Thus, various embodiments of the present invention involve monitoring enzymatic activity by measuring one or more physical properties of the product after having flowed through the lipase, adjusting flow rate, column residence time, or temperature of the initial substrate, or purification of media-processed substrate, and adjusting the process parameters or the amount and type of the purification medium in response to changes in the physical properties in order to increase or improve the enzymatic productivity of the lipase and/or to increase the proportion of esterified, transesterified or interesterified fats or oils in the product so that fats and oils with a desired degree of esterification, interesterification, or transesterification can be produced, particularly those having a substantially uniform increased proportion of esterification, interesterification, or transesterification relative to the initial substrate.

The esterified, transesterified or interesterified product can also be subjected to usual oil refining processes including, but not limited to, refining, bleaching, fractionation, separation or purification process, or additional deodorization processing. The product of the present invention can be separated from any free fatty acid or other by-products by refining techniques well known in the art. In the case of batch slurry type methods, the desired product can be separated using a suitable solvent such as hexane, removing the fatty acid material with an alkali, dehydrating and drying the solvent layer, and removing the solvent from the layer. The desired product can be purified, for example, by column chromatography. The desired products thus obtained are usable for a wide variety of culinary applications.

The following examples show the effect of the substrate pretreatment on the enzyme productivity.

### EXAMPLES

The examples described herein show that productivity of the enzymatic transesterification or esterification is improved by purification of the substrate oil. The following examples are exemplary only and are not intended to limit the scope of the invention as defined by the appended claims.

### Example 1

9.4 g of enzyme (TL IM, Immobilized Thermomyces lanuginosa lipase from Novozymes) was packed in a 1.5 cm diameter jacketed column (30 cm long) at a height of 11.8 cm, which gave 20.8 ml enzyme bed volume. The water circulating through the column jacket was held at 70° C. Substrate oil was made up with liquid oil which had undergone the extraction from an oilseed, degumming, alkali refining, and bleaching steps of conventional soybean oil refining (RB) and fully hydrogenated soybean oil (80/20 by weight) and introduced to the top of the column using an HPLC pump to feed the substrate. The HPLC pump and feed lines were wrapped with heating tape and covered with insulation to prevent any solidification of substrate. The extent of enzyme reaction was monitored by the change of melting properties of the substrate and products, measured as Mettler Drop Point (MDP) as disclosed in U.S. Application Publ. No. 2003/0054509 A1. The substrate blend was pumped to the column at a rate which gave the desired Mettler Drop Point (105-107° F.) of oil exiting the column, and the pumping rate was adjusted during tests to compensate for loss of lipase activity.

Substrate oil was made up with liquid oil which had undergone the degumming, alkali refining and bleaching steps of conventional soybean oil refining (RB) and fully hydrogenated soybean oil. Precolumns comprising granular clay (1.5 volumes granular clay/volume of lipase) were prepared by depositing a layer of granular clay (Agsorb 30/60 LVM-GA, OilDri. Corp., Vernon Hills, Ill.; special run, gas-dried) on top of the layer of lipase in the jacketed column. The precolumn comprising granular clay and TVP was prepared by depositing a layer of TVP (3 volumes TVP/volume lipase) on top of the lipase, and a layer of granular clay (3 volumes granular clay/volume of lipase) on top of the TVP. Substrate oil was passed through the layers comprising the precolumn, which was also heated to 70° C. In one case, the substrate was covered with a layer of nitrogen (nitrogen blanket).

TABLE 1
All substrate oils contained 20% fully hydrogenated
soybean oil and 80% RB soybean oil.

| | Half-life | Lipase productivity |
|---|---|---|
| Treatment | (days) | (g treated oil/g lipase) |
| Control | 14 | 1417 |
| Granular Clay (1.5 vol) | 40 | 3460 |
| Granular Clay (1.5 vol) | 44 | 3705 |
| Nitrogen blanket | | |
| Granular Clay (3 vol) | 73 | 5651 |
| Plus TVP (3 vol) | | |

When lipase was used to interesterify RB soybean oil and fully hydrogenated soybean oil, the half life of the lipase was 14 days, with lipase productivity of 1417 grams of interesterified oil (treated oil) per gram of lipase (enzyme). By pretreating the oil substrate by passing it through a bed of granular clay, the half-life of the lipase was extended and the productivity was more than doubled. Further increase in productivity was obtained by treating the substrate with nitrogen. When substrate was passed through a bed comprising a combination of granular clay and protein (TVP), the half life was more than 5 times longer that the control, and the productivity was increased fourfold.

### Example 2

A substrate mixture comprising refined, bleached, deodorized canola oil and deodorized palm stearin (80% canola oil/20% palm stearin) was made up and passed through a bed of Special run Agsorb 30/60 LVM-GA granular clay (3 volumes granular clay/volume of lipase) before passing through a lipase column in substantially the same manner as described in example 1. A control without granular clay pretreatment was also run. The results are given in Table 2.

TABLE 2
Half life and productivity of lipase used to interesterify
canola oil with deodorized palm olein.

| | Half-life | Lipase productivity |
|---|---|---|
| Treatment | (days) | (g treated oil/g lipase) |
| Control | 7 | 716 |
| Granular Clay (3 vol) | 14 | 1199 |

A substantial improvement in half-life and productivity was obtained by passing the substrate through a bed of granular clay before interesterification.

### Example 3

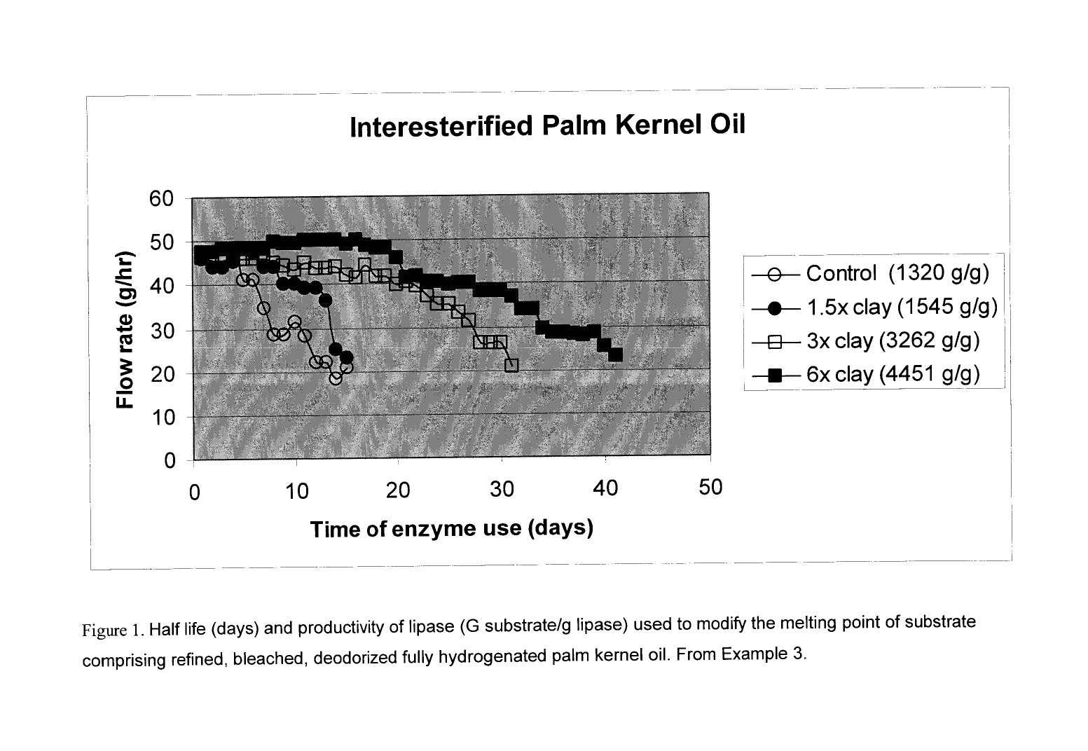

A substrate mixture comprising refined, bleached, deodorized fully hydrogenated palm kernel oil was made up and passed through granular clay (Special run Agsorb 30/60 LVM-GA) beds of varying volume before passing through a lipase column to modify the melting point of the oil in substantially the same manner as described in example 1. A control without granular clay pretreatment was also run. The results are given in Table 3 and FIG. 1.

TABLE 3
Half life and productivity of lipase used to modify
the melting point of comprising refined, bleached,
deodorized fully hydrogenated palm kernel oil.

| | Half-life | Lipase productivity |
|---|---|---|
| Treatment | (days) | (g treated oil/g lipase) |
| Control | 15 | 1320 |
| Granular Clay (1.5 vol) | 14 | 1546 |
| Granular Clay (3 vol) | 31 | 3262 |
| Granular Clay (6 vol) | 41 | 4451 |

Substantial improvements in half-life and productivity were obtained by passing the substrate through a bed of granular clay before interesterification. Improvements increased as the volume of pretreatment bed of granular clay increased.

### Example 4

Substrate was prepared as in Example 3 and passed through a precolumn containing 3 volumes of food-compatible granular clay (Special run Agsorb 30/60 LVM-GA) per volume of lipase in substantially the same manner as described in Example 3. However, at intervals the granular clay was removed and fresh granular clay was added to replace the granular clay. The clay was replaced on days 13, 25, 36, 52, 66, 82, 95, and 111. The half-life of the lipase was reached at 150 days, with a productivity value of 9,673 grams of oil/gram of lipase preparation.

### Example 5

Substrate was prepared as in Example 1 and passed through granular clay (Special run Agsorb 30/60 LVM-GA) beds of varying volume before passing the substrate through a lipase column to modify the melting point of the oil in the substrate in substantially the same manner as described in Example 1. Two controls without granular clay pretreatment were also run. In addition, the substrate mixture was passed through 3 bed volumes of granular carbon (Cal®12×40, Granular bituminous coal-based carbon from Calgon Carbon Corp. Pittsburgh, Pa.) The results are given in Table 4.

TABLE 4
Half life and productivity of lipase used to
modify substrate oils contained 20% fully hydrogenated
soybean oil and 80% RB soybean oil.

| | Half-life | Lipase productivity |
|---|---|---|
| Treatment | (days) | (g treated oil/g lipase) |
| Control (no treatment) | 21 | 1692 |
| Control (no treatment), repeat | 14 | 1264 |
| Granular Clay, 3 BV | 31 | 2586 |
| Granular Clay, 3 BV, repeat | 39 | 3042 |
| Granular Clay, 3 BV and 3 BV | 43 | 3447 |
| Granular Carbon* | | |

Substantial improvements in half-life and productivity were obtained by passing the substrate through a bed of granular clay before interesterification. Passing the substrate through a bed of a combination of granular clay and granular carbon gave even more improvement in lipase half-life and productivity.

While the foregoing invention has been described in some detail for purposes of clarity and understanding, it will be appreciated by one skilled in the art from a reading of this disclosure that various changes in form and detail can be made without departing from the true scope of the invention and appended claims. All publications mentioned above are hereby incorporated in their entirety by reference.

What is claimed is:

1. A process for producing fats or oils comprising:
placing a glyceride in contact with a compound selected from the group consisting of granular clay, granular carbon, and a combination thereof, thus forming a purified substrate; and
placing the purified substrate in contact with a lipase, thus producing the fat or the oil.

2. The process of claim 1, wherein at least 90 percent of the granular clay has particles greater than 80 microns in size.

3. (canceled)

4. The process of claim 1, wherein at least 95 percent of the granular clay has particles ranging from 20-60 mesh size.

5. The process of claim 1, wherein the granular clay has a moisture content of less than 5%.

6. (canceled)

7. The process of claim 1, further comprising placing a compound selected from the group consisting of free fatty acids, fatty acids, monohydroxyl alcohols, polyhydroxyl alcohols, esters thereof, and combinations of any thereof in contact with the granular clay, granular carbon, or a combination thereof.

8. The process of claim 1, wherein the glyceride is selected from the group consisting of butterfat, cocoa butter, cocoa butter substitutes, illipe fat, kokum butter, milk fat, mowrah fat, phulwara butter, sal fat, shea fat, borneo tallow, lard, lanolin, beef tallow, mutton tallow, tallow, animal fat, canola oil, castor oil, coconut oil, coriander oil, corn oil, cottonseed oil, hazelnut oil, hempseed oil, jatropha oil, linseed oil, mango kernel oil, meadowfoam oil, mustard oil, neat's foot oil, olive oil, palm oil, palm kernel oil, peanut oil, rapeseed oil, rice bran oil, safflower oil, sasanqua oil, shea butter, soybean oil, sunflower seed oil, tall oil, tsubaki oil, vegetable oils, marine oils which can be converted into plastic fats, marine oils which can be converted into solid fats, menhaden oil, candlefish oil, cod-liver oil, orange roughy oil, pile herd oil, sardine oil, whale oils, herring oils, 1,3-dipalmitoyl-2-monooleine (POP), 1(3)-palmitoyl-3(1)-stearoyl-2-monooleine (POSt), 1,3-distearoyl-2-monooleine (StOSt), triglyceride, diglyceride, 1,3-diglycerides, monoglyceride, behenic acid triglyceride, triolein, tripalmitin, tristearin, palm olein, palm stearin, palm kernel olein, palm kernel stearin, triglycerides of medium chain fatty acids, processed partially hydrogenated oils of any thereof, processed fully hydrogenated oils of any thereof; fractionated oils of any thereof, partially hydrogenated soybean oil, partially hydrogenated corn oil, partially hydrogenated cottonseed oil, fully hydrogenated soybean oil, fully hydrogenated corn oil, partially hydrogenated palm oil, Partially hydrogenated palm kernel oil, fully hydrogenated palm oil, fully hydrogenated palm kernel oil, fractionated palm oil, fractionated palm kernel oil, fractionated partially hydrogenated palm oil, fractionated Partially hydrogenated palm kernel oil, and combinations of any thereof.

9. The process of claim 1, wherein the esters are selected from the group consisting of wax esters, alkyl esters, methyl esters, ethyl esters, isopropyl esters, octadecyl esters, aryl esters, propylene glycol esters, ethylene glycol esters, 1,2-propanediol esters, 1,3-propanediol esters, and any combination thereof.

10. The process of claim 1, further comprising placing a primary, secondary or tertiary monohydroxyl or polyhydroxyl alcohols of annular, straight or branched chain compounds in contact with the granular clay, granular carbon, or a combination thereof.

11. The process of claim 1, further comprising packing the granular clay, granular carbon, or a combination thereof and the lipase in a column.

12. The process of claim 1, further comprising mixing the granular clay, granular carbon, or a combination thereof in a tank for a batch process or mixing the granular clay, granular carbon, or a combination thereof in a series of tanks for a series of batch processes.

13. The process of claim 12, further comprising mixing the lipase in the tank for the batch process or flowing the glyceride through a column containing the lipase.

14-15. (canceled)

16. The process of claim 1, further comprising an act selected from the group consisting of: adjusting a duration of time that glyceride contacts the lipase; adjusting a temperature of the glyceride, the granular clay, granular carbon, or a combination thereof, the lipase, or any combination thereof; and a combination thereof.

17-30. (canceled)

31. The process of claim 1, wherein an enzymatic activity half-life of the lipase is more than 2.5 times greater than the enzymatic activity half-life of a lipase contacted with a glyceride not placed in contact with the granular clay, granular carbon, or any combinations thereof.

32-42. (canceled)

43. A system for treating a lipid, comprising:
a container configured to place a lipid in contact with a substance capable of extending a half-life of an enzyme, the container comprising:
the substance capable of extending the half-life of the enzyme; and
the lipid.

44. The system of claim 43, wherein the container further comprises:
an inlet for introducing the lipid into the container; and
an outlet for allowing the lipid to exit the container.

45. The system of claim 43, further comprising:
a second container configured to place the lipid in contact with the enzyme, the second container comprising:
the enzyme; and
the lipid.

46. (canceled)

47. The system of claim 45, further comprising a conduit configured to transport the lipid from the container to the second container.

48. The system of claim 43, wherein the substance capable of extending the half-life of the enzyme is selected from the group consisting of a granular clay, a granular carbon, and any combination thereof.

49. The system of claim 43, wherein the substance capable of extending the half-life of the enzyme is selected from the group consisting of a food grade granular clay, a granular carbon, and any combinations thereof.

50. The system of claim 43, wherein the enzyme is a lipase.

51-53. (canceled)

54. A composition produced by a process comprising:
placing an ingestible substance in contact with a substance selected from the group consisting of granular clay, granular clay suitable for contact with human food products, food grade granular clay, food-compatible granular clay, granular clay approved for use in the production of human food products, a combination of granular clay and protein, a combination of granular clay and granular carbon, and any combinations thereof.
